(19) United States
(12) Patent Application Publication
(10) Pub. No.: US 2012/0277482 A1
(43) Pub. Date: Nov. 1, 2012

(54) Removing Water from an Acetic Acid Stream in the Production of Alcohols

(75) Inventors: David LEE, Seabrook, TX (US); Adam OROSCO, Houston, TX (US); Lincoln SARAGER, Houston, TX (US); R. Jay WARNER, Houston, TX (US); Trinity HORTON, Houston, TX (US); Radmila JEVTIC, Houston, TX (US); Victor J. JOHNSTON, Houston, TX (US)

(73) Assignee: CELANESE INTERNATIONAL CORPORATION, Dallas, TX (US)

(21) Appl. No.: 13/094,675

(22) Filed: Apr. 26, 2011

(51) Int. Cl. C07C 29/149

(52) U.S. Cl. 568/884

(57) ABSTRACT

A process for removing water from an acetic acid stream in the production of alcohols, such as ethanol produced by hydrogenating acetic acid. Water is removed from the acetic acid stream using an adsorption unit, membrane, molecular sieves, or a combination thereof, to form a return stream. The return stream may be recycled to the hydrogenation reactor.

### FIELD OF THE INVENTION

The present invention relates generally to processes for producing alcohols obtained by hydrogenating acetic acid and, in particular, to a process for removing water from an acetic acid stream to form a return stream, which may be recycled to the reactor.

### BACKGROUND OF THE INVENTION

Ethanol for industrial use is conventionally produced from petrochemical feed stocks, such as oil, natural gas, or coal, from feed stock intermediates, such as syngas, or from starchy materials or cellulose materials, such as corn or sugar cane. Conventional methods for producing ethanol from petrochemical feed stocks, as well as from cellulose materials, include the acid-catalyzed hydration of ethylene, methanol homologation, direct alcohol synthesis, and Fischer-Tropsch synthesis. Instability in petrochemical feed stock prices contributes to fluctuations in the cost of conventionally produced ethanol, making the need for alternative sources of ethanol production all the greater when feed stock prices rise. Starchy materials, as well as cellulose material, are converted to ethanol by fermentation. However, fermentation is typically used for consumer production of ethanol, which is suitable for fuels or human consumption. In addition, fermentation of starchy or cellulose materials competes with food sources and places restraints on the amount of ethanol that can be produced for industrial use.

Ethanol production via the reduction of alkanoic acids and/or other carbonyl group-containing compounds has been widely studied, and a variety of combinations of catalysts, supports, and operating conditions have been mentioned in the literature. During the reduction of alkanoic acid, e.g., acetic acid, other compounds are formed with ethanol or are formed in side reactions. These impurities limit the production and recovery of ethanol from such reaction mixtures. For example, during hydrogenation, esters are produced that together with ethanol and/or water form azeotropes, which are difficult to separate. In addition when conversion is incomplete, unreacted acetic acid remains in the crude ethanol product, which must be removed to recover ethanol.

EP02060553 describes a process for converting hydrocarbons to ethanol involving converting the hydrocarbons to ethanoic acid and hydrogenating the ethanoic acid to ethanol. The stream from the hydrogenation reactor is separated to obtain an ethanol stream and a stream of acetic acid and ethyl acetate, which is recycled to the hydrogenation reactor.

The need remains for improved processes for recovering ethanol from a crude product obtained by reducing alkanoic acids, such as acetic acid, and/or other carbonyl group-containing compounds.

### SUMMARY OF THE INVENTION

In a first embodiment, the present invention is directed to a process for producing ethanol, comprising hydrogenating acetic acid in a reactor to form a crude ethanol product comprising ethanol, acetic acid, water and ethyl acetate; separating a portion of the crude ethanol product to yield a first stream comprising ethanol, ethyl acetate, and water, and a second stream comprising acetic acid and water; removing water from the second stream to yield a return stream comprising acetic acid and less than 15 wt. % water; and recovering ethanol from the first stream. A portion of the return stream is fed to the reactor to produce additional ethanol. In other aspects, a portion of the return stream may be used in the production of vinyl acetate, acetic anhydride, or ethyl acetate.

In a second embodiment, the present invention is directed to a process for producing ethanol, comprising hydrogenating acetic acid to form a crude ethanol product; separating a portion of the crude ethanol product in a first distillation column to yield a first distillate comprising ethanol, ethyl acetate, and water; and a first residue comprising acetic acid; removing water from the first residue to form a return stream comprising acetic acid; and separating a portion of the first distillate in a second distillation column to yield a second residue comprising ethanol and a second distillate comprising ethyl acetate.

In a third embodiment, the present invention is direct to a process for producing ethanol, comprising providing a crude ethanol product comprising ethanol, acetic acid, ethyl acetate, and water; separating a portion of the crude ethanol product to yield a first stream comprising ethanol, ethyl acetate, and water, and a second stream comprising acetic acid and water; removing water from the second stream to yield a return stream comprising acetic acid and less than 15 wt. % water; and recovering ethanol from the first stream.

In a fourth embodiment, the present invention is directed to a process for producing ethanol, comprising hydrogenating acetic acid in a reactor to form a crude ethanol product comprising ethanol, acetic acid, water and ethyl acetate; separating a portion of the crude ethanol product in a first column to yield a first distillate comprising ethanol, ethyl acetate, and water, and a first residue comprising acetic acid and water; removing water from the first residue to yield a return stream comprising acetic acid and less than 15 wt. % water; and recovering ethanol from the first distillate.

### BRIEF DESCRIPTION OF DRAWINGS

The invention may be more completely understood in consideration of the following detailed description of various embodiments of the invention in connection with the accompanying drawings, wherein like numerals designate similar parts.

FIG. 1 is a schematic diagram of an ethanol production system with a water separator for removing water from an acetic acid stream in accordance with one embodiment of the present invention.

### DETAILED DESCRIPTION OF THE INVENTION

### Introduction

The present invention relates to processes for recovering ethanol produced by hydrogenating acetic acid in the presence of a catalyst. The hydrogenation reaction produces a crude ethanol product that comprises ethanol, water, ethyl acetate, unreacted acetic acid, and other impurities. The concentration of unreacted acetic acid in the crude ethanol product may depend on the catalyst composition and process conditions for the hydrogenation. In one embodiment, it is advantageous to recover an acetic acid stream comprising acetic acid and water from the crude ethanol product. Preferably the acetic acid stream is recovered as a residue from a first separation column. Water is removed from the acetic acid stream. The resulting return stream may be recycled to the hydrogenation reactor to produce additional ethanol. In other embodiments, the resulting return stream may be fed to other production facilities including facilities for producing vinyl acetate, acetic anhydride, and ethyl acetate. The acetic acid in the return stream may also be used as a separate product for other applications.

In one embodiment of the invention, the process involves introducing the crude ethanol product to an initial separation column (first column), which separates the crude ethanol product into a distillate (first distillate) comprising ethanol, ethyl acetate, and optionally water, and a residue (first residue), e.g., acetic acid stream, comprising unreacted acetic acid and water. The ethanol product may be recovered from the distillate. Preferably, a substantial portion of the water is removed in the residue so that further water separation from the distillate is not necessary. This separation scheme may advantageously result in improved ethanol production efficiency using less energy compared to other separation schemes. The present invention preferably further comprises the steps of separating water from the acetic acid stream, and recycling the acetic acid to the hydrogenation process.

The concentration of acetic acid in the acetic acid stream may vary depending on factors such as the acetic acid conversion in the hydrogenation reactor and the separation conditions maintained in the initial separation column. In one embodiment, the acetic acid stream comprises at least 2 wt. % acetic acid, e.g., at least 3 wt. % or at least 26 wt. % acetic acid. In terms of ranges, the acetic acid stream may comprise from 2.5 wt. % to 90 wt. % acetic acid, e.g., from 2.5 wt. % to 28 wt. %, or from 80 wt. % to 90 wt. % acetic acid. In addition, the amount of water in the acetic acid stream may vary, but generally the acetic acid stream comprises less than 98 wt. % water, less than 97 wt. % water or less than 74 wt. % water. In terms of ranges, the acetic acid stream may comprise from 10 wt. % to 97.5 wt. % water, e.g., from 72 wt. % to 97.5 wt. %, or from 10 wt. % to 20 wt. % water.

The processes of the present invention may employ any suitable technique for removing water from the acetic acid stream. The water may be removed in the liquid or vapor phase. Suitable water separators should be able to tolerate acidic conditions. A membrane or an array of membranes may be employed to separate water from the residue. The membrane or array of membranes may be selected from any suitable membrane that are acid resistant. The membranes may be capable of permeating water or acetic acid.

In preferred embodiments, at least 50% of the water in the acetic acid stream is removed, e.g., at least 60% of the water or at least 75 wt. % of the water, based on the total amount of water in the acetic acid stream. Depending on the separation efficiency of the water separator removal of more than 99% of the water may be possible. In more preferred embodiments, from 90 to 99% of the water may be removed from the acetic acid stream. The water stream may be purged from the system. The resulting acetic acid, e.g., return stream, may comprise only a minor amount of water, e.g., less than 15 wt. %, less than 10 wt. %, or less than 5 wt. % water. In terms of ranges return stream may comprise from 0.01 wt. % to 15 wt. % water, e.g., from 0.01 wt. % to 10 wt. % or from 0.1 wt. % to 5 wt. %. The resulting acetic acid return stream may be returned to the hydrogenation reactor to produce additional ethanol or used in other production facilities for vinyl acetate, acetic anhydride, or ethyl acetate.

### Hydrogenation of Acetic Acid

The process of the present invention may be used with any hydrogenation process for producing ethanol The materials, catalysts, reaction conditions, and separation processes that may be used in the hydrogenation of acetic acid are described further below.

The raw materials, acetic acid and hydrogen, used in connection with the process of this invention may be derived from any suitable source including natural gas, petroleum, coal, biomass, and so forth. As examples, acetic acid may be produced via methanol carbonylation, acetaldehyde oxidation, ethylene oxidation, oxidative fermentation, and anaerobic fermentation. Methanol carbonylation processes suitable for production of acetic acid are described in U.S. Pat. Nos. 7,208,624; 7,115,772; 7,005,541; 6,657,078; 6,627,770; 6,143,930; 5,599,976; 5,144,068; 5,026,908; 5,001,259; and 4,994,608, the entire disclosures of which are incorporated herein by reference. Optionally, the production of ethanol may be integrated with such methanol carbonylation processes.

As petroleum and natural gas prices fluctuate becoming either more or less expensive, methods for producing acetic acid and intermediates such as methanol and carbon monoxide from alternate carbon sources have drawn increasing interest. In particular, when petroleum is relatively expensive, it may become advantageous to produce acetic acid from synthesis gas (“syngas”) that is derived from more available carbon sources. U.S. Pat. No. 6,232,352, the entirety of which is incorporated herein by reference, for example, teaches a method of retrofitting a methanol plant for the manufacture of acetic acid. By retrofitting a methanol plant, the large capital costs associated with CO generation for a new acetic acid plant are significantly reduced or largely eliminated. All or part of the syngas is diverted from the methanol synthesis loop and supplied to a separator unit to recover CO, which is then used to produce acetic acid. In a similar manner, hydrogen for the hydrogenation step may be supplied from syngas.

In some embodiments, some or all of the raw materials for the above-described acetic acid hydrogenation process may be derived partially or entirely from syngas. For example, the acetic acid may be formed from methanol and carbon monoxide, both of which may be derived from syngas. The syngas may be formed by partial oxidation reforming or steam reforming, and the carbon monoxide may be separated from syngas. Similarly, hydrogen that is used in the step of hydrogenating the acetic acid to form the crude ethanol product may be separated from syngas. The syngas, in turn, may be derived from variety of carbon sources. The carbon source, for example, may be selected from the group consisting of natural gas, oil, petroleum, coal, biomass, and combinations thereof. Syngas or hydrogen may also be obtained from bio-derived methane gas, such as bio-derived methane gas produced by landfills or agricultural waste.

In another embodiment, the acetic acid used in the hydrogenation step may be formed from the fermentation of biomass. The fermentation process preferably utilizes an acetogenic process or a homoacetogenic microorganism to ferment sugars to acetic acid producing little, if any, carbon dioxide as a by-product. The carbon efficiency for the fermentation process preferably is greater than 70%, greater than 80% or greater than 90% as compared to conventional yeast processing, which typically has a carbon efficiency of about 67%. Optionally, the microorganism employed in the fermentation process is of a genus selected from the group consisting of Clostridium, Lactobacillus, Moorella, Thermoanaerobacter, Propionibacterium, Propionispera, Anaerobiospirillum, and Bacteriodes, and in particular, species selected from the group consisting of Clostridium formicoaceticum, Clostridium butyricum, Moorella thermoacetica, Thermoanaerobacter kivui, Lactobacillus delbrukii, Propionibacterium acidipropionici, Propionispera arboris, Anaerobiospirillum succinicproducens, Bacteriodes amylophilus and Bacteriodes ruminicola. Optionally in this process, all or a portion of the unfermented residue from the biomass, e.g., lignans, may be gasified to form hydrogen that may be used in the hydrogenation step of the present invention. Exemplary fermentation processes for forming acetic acid are disclosed in U.S. Pat. Nos. 6,509,180; 6,927,048; 7,074,603; 7,507,562; 7,351,559; 7,601,865; 7,682,812; and 7,888,082, the entireties of which are incorporated herein by reference. See also U.S. Pub. Nos. 2008/0193989 and 2009/0281354, the entireties of which are incorporated herein by reference.

Examples of biomass include, but are not limited to, agricultural wastes, forest products, grasses, and other cellulosic material, timber harvesting residues, softwood chips, hardwood chips, tree branches, tree stumps, leaves, bark, sawdust, off-spec paper pulp, corn, corn stover, wheat straw, rice straw, sugarcane bagasse, switchgrass, miscanthus, animal manure, municipal garbage, municipal sewage, commercial waste, grape pumice, almond shells, pecan shells, coconut shells, coffee grounds, grass pellets, hay pellets, wood pellets, cardboard, paper, plastic, and cloth. See, e.g., U.S. Pat. No. 7,884,253, the entirety of which is incorporated herein by reference. Another biomass source is black liquor, a thick, dark liquid that is a byproduct of the Kraft process for transforming wood into pulp, which is then dried to make paper. Black liquor is an aqueous solution of lignin residues, hemicellulose, and inorganic chemicals.

U.S. Pat. No. RE 35,377, also incorporated herein by reference, provides a method for the production of methanol by conversion of carbonaceous materials such as oil, coal, natural gas and biomass materials. The process includes hydrogasification of solid and/or liquid carbonaceous materials to obtain a process gas which is steam pyrolized with additional natural gas to form synthesis gas. The syngas is converted to methanol which may be carbonylated to acetic acid. The method likewise produces hydrogen which may be used in connection with this invention as noted above. U.S. Pat. No. 5,821,111, which discloses a process for converting waste biomass through gasification into synthesis gas, and U.S. Pat. No. 6,685,754, which discloses a method for the production of a hydrogen-containing gas composition, such as a synthesis gas including hydrogen and carbon monoxide, are incorporated herein by reference in their entireties.

The acetic acid fed to the hydrogenation reaction may also comprise other carboxylic acids and anhydrides, as well as acetaldehyde and acetone. Preferably, a suitable acetic acid feed stream comprises one or more of the compounds selected from the group consisting of acetic acid, acetic anhydride, acetaldehyde, ethyl acetate, and mixtures thereof. These other compounds may also be hydrogenated in the processes of the present invention. In some embodiments, the presence of carboxylic acids, such as propanoic acid or its anhydride, may be beneficial in producing propanol. Water may also be present in the acetic acid feed.

Alternatively, acetic acid in vapor form may be taken directly as crude product from the flash vessel of a methanol carbonylation unit of the class described in U.S. Pat. No. 6,657,078, the entirety of which is incorporated herein by reference. The crude vapor product, for example, may be fed directly to the ethanol synthesis reaction zones of the present invention without the need for condensing the acetic acid and light ends or removing water, saving overall processing costs.

The acetic acid may be vaporized at the reaction temperature, following which the vaporized acetic acid may be fed along with hydrogen in an undiluted state or diluted with a relatively inert carrier gas, such as nitrogen, argon, helium, carbon dioxide and the like. For reactions run in the vapor phase, the temperature should be controlled in the system such that it does not fall below the dew point of acetic acid. In one embodiment, the acetic acid may be vaporized at the boiling point of acetic acid at the particular pressure, and then the vaporized acetic acid may be further heated to the reactor inlet temperature. In another embodiment, the acetic acid is mixed with other gases before vaporizing, followed by heating the mixed vapors up to the reactor inlet temperature. Preferably, the acetic acid is transferred to the vapor state by passing hydrogen and/or recycle gas through the acetic acid at a temperature at or below 125° C., followed by heating of the combined gaseous stream to the reactor inlet temperature.

Some embodiments of the process of hydrogenating acetic acid to form ethanol may include a variety of configurations using a fixed bed reactor or a fluidized bed reactor. In many embodiments of the present invention, an “adiabatic” reactor can be used; that is, there is little or no need for internal plumbing through the reaction zone to add or remove heat. In other embodiments, a radial flow reactor or reactors may be employed, or a series of reactors may be employed with or with out heat exchange, quenching, or introduction of additional feed material. Alternatively, a shell and tube reactor provided with a heat transfer medium may be used. In many cases, the reaction zone may be housed in a single vessel or in a series of vessels with heat exchangers therebetween.

In preferred embodiments, the catalyst is employed in a fixed bed reactor, e.g., in the shape of a pipe or tube, where the reactants, typically in the vapor form, are passed over or through the catalyst. Other reactors, such as fluid or ebullient bed reactors, can be employed. In some instances, the hydrogenation catalysts may be used in conjunction with an inert material to regulate the pressure drop of the reactant stream through the catalyst bed and the contact time of the reactant compounds with the catalyst particles.

The hydrogenation reaction may be carried out in either the liquid phase or vapor phase. Preferably, the reaction is carried out in the vapor phase under the following conditions. The reaction temperature may range from 125° C. to 350° C., e.g., from 200° C. to 325° C., from 225° C. to 300° C., or from 250° C. to 300° C. The pressure may range from 10 kPa to 3000 kPa, e.g., from 50 kPa to 2300 kPa, or from 100 kPa to 1500 kPa. The reactants may be fed to the reactor at a gas hourly space velocity (GHSV) of greater than 500 hr⁻¹, e.g., greater than 1000 hr⁻¹, greater than 2500 hr⁻¹ or even greater than 5000 hr⁻¹. In terms of ranges the GHSV may range from 50 hr⁻¹ to 50,000 hr⁻¹, e.g., from 500 hr⁻¹ to 30,000 hr⁻¹, from 1000 hr⁻¹ to 10,000 hr⁻¹, or from 1000 hr⁻¹ to 6500 hr⁻¹.

The hydrogenation optionally is carried out at a pressure just sufficient to overcome the pressure drop across the catalytic bed at the GHSV selected, although there is no bar to the use of higher pressures, it being understood that considerable pressure drop through the reactor bed may be experienced at high space velocities, e.g., 5000 hr⁻¹ or 6,500 hr⁻¹.

Although the reaction consumes two moles of hydrogen per mole of acetic acid to produce one mole of ethanol, the actual molar ratio of hydrogen to acetic acid in the feed stream may vary from about 100:1 to 1:100, e.g., from 50:1 to 1:50, from 20:1 to 1:2, or from 12:1 to 1:1. Most preferably, the molar ratio of hydrogen to acetic acid is greater than 2:1, e.g., greater than 4:1 or greater than 8:1.

Contact or residence time can also vary widely, depending upon such variables as amount of acetic acid, catalyst, reactor, temperature, and pressure. Typical contact times range from a fraction of a second to more than several hours when a catalyst system other than a fixed bed is used, with preferred contact times, at least for vapor phase reactions, of from 0.1 to 100 seconds, e.g., from 0.3 to 80 seconds or from 0.4 to 30 seconds.

The hydrogenation of acetic acid to form ethanol is preferably conducted in the presence of a hydrogenation catalyst. Suitable hydrogenation catalysts include catalysts comprising a first metal and optionally one or more of a second metal, a third metal or any number of additional metals, optionally on a catalyst support. The first and optional second and third metals may be selected from Group IB, IIB, IIIB, IVB, VB, VIIB, VIIB, VIII transition metals, a lanthanide metal, an actinide metal or a metal selected from any of Groups IIIA, IVA, VA, and VIA. Preferred metal combinations for some exemplary catalyst compositions include platinum/tin, platinum/ruthenium, platinum/rhenium, palladium/ruthenium, palladium/rhenium, cobalt/palladium, cobalt/platinum, cobalt/chromium, cobalt/ruthenium, cobalt/tin, silver/palladium, copper/palladium, copper/zinc, nickel/palladium, gold/palladium, ruthenium/rhenium, and ruthenium/iron. Exemplary catalysts are further described in U.S. Pat. No. 7,608,744 and U.S. Pub. No. 2010/0029995, the entireties of which are incorporated herein by reference. In another embodiment, the catalyst comprises a Co/Mo/S catalyst of the type described in U.S. Pub. No. 2009/0069609, the entirety of which is incorporated herein by reference.

In one embodiment, the catalyst comprises a first metal selected from the group consisting of copper, iron, cobalt, nickel, ruthenium, rhodium, palladium, osmium, iridium, platinum, titanium, zinc, chromium, rhenium, molybdenum, and tungsten. Preferably, the first metal is selected from the group consisting of platinum, palladium, cobalt, nickel, and ruthenium. More preferably, the first metal is selected from platinum and palladium. In embodiments of the invention where the first metal comprises platinum, it is preferred that the catalyst comprises platinum in an amount less than 5 wt. %, e.g., less than 3 wt. % or less than 1 wt. %, due to the high commercial demand for platinum.

As indicated above, in some embodiments, the catalyst further comprises a second metal, which typically would function as a promoter. If present, the second metal preferably is selected from the group consisting of copper, molybdenum, tin, chromium, iron, cobalt, vanadium, tungsten, palladium, platinum, lanthanum, cerium, manganese, ruthenium, rhenium, gold, and nickel. More preferably, the second metal is selected from the group consisting of copper, tin, cobalt, rhenium, and nickel. More preferably, the second metal is selected from tin and rhenium.

In certain embodiments where the catalyst includes two or more metals, e.g., a first metal and a second metal, the first metal is present in the catalyst in an amount from 0.1 to 10 wt. %, e.g., from 0.1 to 5 wt. %, or from 0.1 to 3 wt. %. The second metal preferably is present in an amount from 0.1 to 20 wt. %, e.g., from 0.1 to 10 wt. %, or from 0.1 to 5 wt. %. For catalysts comprising two or more metals, the two or more metals may be alloyed with one another or may comprise a non-alloyed metal solution or mixture.

The preferred metal ratios may vary depending on the metals used in the catalyst. In some exemplary embodiments, the mole ratio of the first metal to the second metal is from 10:1 to 1:10, e.g., from 4:1 to 1:4, from 2:1 to 1:2, from 1.5:1 to 1:1.5 or from 1.1:1 to 1:1.1.

The catalyst may also comprise a third metal selected from any of the metals listed above in connection with the first or second metal, so long as the third metal is different from the first and second metals. In preferred aspects, the third metal is selected from the group consisting of cobalt, palladium, ruthenium, copper, zinc, platinum, tin, and rhenium. More preferably, the third metal is selected from cobalt, palladium, and ruthenium. When present, the total weight of the third metal preferably is from 0.05 to 4 wt. %, e.g., from 0.1 to 3 wt. %, or from 0.1 to 2 wt. %.

In addition to one or more metals, in some embodiments of the present invention the catalysts further comprise a support or a modified support. As used herein, the term “modified support” refers to a support that includes a support material and a support modifier, which adjusts the acidity of the support material.

The total weight of the support or modified support, based on the total weight of the catalyst, preferably is from 75 to 99.9 wt. %, e.g., from 78 to 97 wt. %, or from 80 to 95 wt. %. In preferred embodiments that utilize a modified support, the support modifier is present in an amount from 0.1 to 50 wt. %, e.g., from 0.2 to 25 wt. %, from 0.5 to 15 wt. %, or from 1 to 8 wt. %, based on the total weight of the catalyst. The metals of the catalysts may be dispersed throughout the support, layered throughout the support, coated on the outer surface of the support (i.e., egg shell), or decorated on the surface of the support.

As will be appreciated by those of ordinary skill in the art, support materials are selected such that the catalyst system is suitably active, selective and robust under the process conditions employed for the formation of ethanol.

Suitable support materials may include, for example, stable metal oxide-based supports or ceramic-based supports. Preferred supports include silicaceous supports, such as silica, silica/alumina, a Group IIA silicate such as calcium metasilicate, pyrogenic silica, high purity silica, and mixtures thereof. Other supports may include, but are not limited to, iron oxide, alumina, titania, zirconia, magnesium oxide, carbon, graphite, high surface area graphitized carbon, activated carbons, and mixtures thereof.

As indicated, the catalyst support may be modified with a support modifier. In some embodiments, the support modifier may be an acidic modifier that increases the acidity of the catalyst. Suitable acidic support modifiers may be selected from the group consisting of: oxides of Group IVB metals, oxides of Group VB metals, oxides of Group VIB metals, oxides of Group VIIB metals, oxides of Group VIIIB metals, aluminum oxides, and mixtures thereof. Acidic support modifiers include those selected from the group consisting of TiO₂, ZrO₂, Nb₂O₅, Ta₂O₅, Al₂O₃, B₂O₃, P₂O₅, and Sb₂O₃. Preferred acidic support modifiers include those selected from the group consisting of TiO₂, ZrO₂, Nb₂O₅, Ta₂O₅, and Al₂O₃. The acidic modifier may also include WO₃, MoO₃, Fe₂O₃, Cr₂O₃, V₂O₅, MnO₂, CuO, Co₂O₃, and Bi₂O₃.

In another embodiment, the support modifier may be a basic modifier that has a low volatility or no volatility. Such basic modifiers, for example, may be selected from the group consisting of: (i) alkaline earth oxides, (ii) alkali metal oxides, (iii) alkaline earth metal metasilicates, (iv) alkali metal metasilicates, (v) Group IIB metal oxides, (vi) Group IIB metal metasilicates, (vii) Group IIIB metal oxides, (viii) Group IIIB metal metasilicates, and mixtures thereof. In addition to oxides and metasilicates, other types of modifiers including nitrates, nitrites, acetates, and lactates may be used. Preferably, the support modifier is selected from the group consisting of oxides and metasilicates of any of sodium, potassium, magnesium, calcium, scandium, yttrium, and zinc, as well as mixtures of any of the foregoing. More preferably, the basic support modifier is a calcium silicate, and even more preferably calcium metasilicate (CaSiO₃). If the basic support modifier comprises calcium metasilicate, it is preferred that at least a portion of the calcium metasilicate is in crystalline form.

A preferred silica support material is SS61138 High Surface Area (HSA) Silica Catalyst Carrier from Saint Gobain N or Pro. The Saint-Gobain N or Pro SS61138 silica exhibits the following properties: contains approximately 95 wt. % high surface area silica; surface area of about 250 m²/g; median pore diameter of about 12 nm; average pore volume of about 1.0 cm³/g as measured by mercury intrusion porosimetry and a packing density of about 0.352 g/cm³ (22 lb/ft³).

A preferred silica/alumina support material is KA-160 silica spheres from Sud Chemie having a nominal diameter of about 5 mm, a density of about 0.562 g/ml, an absorptivity of about 0.583 g H₂O/g support, a surface area of about 160 to 175 m²/g, and a pore volume of about 0.68 ml/g.

The catalyst compositions suitable for use with the present invention preferably are formed through metal impregnation of the modified support, although other processes such as chemical vapor deposition may also be employed. Such impregnation techniques are described in U.S. Pat. Nos. 7,608,744 and 7,863,489 and U.S. Pub. No. 2010/0197485 referred to above, the entireties of which are incorporated herein by reference.

In particular, the hydrogenation of acetic acid may achieve favorable conversion of acetic acid and favorable selectivity and productivity to ethanol. For purposes of the present invention, the term “conversion” refers to the amount of acetic acid in the feed that is converted to a compound other than acetic acid. Conversion is expressed as a mole percentage based on acetic acid in the feed. The conversion may be at least 10%, e.g., at least 20%, at least 40%, at least 50%, at least 60%, at least 70% or at least 80%. Although catalysts that have high conversions are desirable, such as at least 80% or at least 90%, in some embodiments a low conversion may be acceptable at high selectivity for ethanol. It is, of course, well understood that in many cases, it is possible to compensate for conversion by appropriate recycle streams or use of larger reactors, but it is more difficult to compensate for poor selectivity.

Selectivity is expressed as a mole percent based on converted acetic acid. It should be understood that each compound converted from acetic acid has an independent selectivity and that selectivity is independent from conversion. For example, if 60 mole % of the converted acetic acid is converted to ethanol, we refer to the ethanol selectivity as 60%. Preferably, the catalyst selectivity to ethoxylates is at least 60%, e.g., at least 70%, or at least 80%. As used herein, the term “ethoxylates” refers specifically to the compounds ethanol, acetaldehyde, and ethyl acetate. Preferably, the selectivity to ethanol is at least 80%, e.g., at least 85% or at least 88%. Preferred embodiments of the hydrogenation process also have low selectivity to undesirable products, such as methane, ethane, and carbon dioxide. The selectivity to these undesirable products preferably is less than 4%, e.g., less than 2% or less than 1%. More preferably, these undesirable products are present in undetectable amounts. Formation of alkanes may be low, and ideally less than 2%, less than 1%, or less than 0.5% of the acetic acid passed over the catalyst is converted to alkanes, which have little value other than as fuel.

The term “productivity,” as used herein, refers to the grams of a specified product, e.g., ethanol, formed during the hydrogenation based on the kilograms of catalyst used per hour. A productivity of at least 100 grams of ethanol per kilogram of catalyst per hour, e.g., at least 400 grams of ethanol per kilogram of catalyst per hour or at least 600 grams of ethanol per kilogram of catalyst per hour, is preferred. In terms of ranges, the productivity preferably is from 100 to 3,000 grams of ethanol per kilogram of catalyst per hour, e.g., from 400 to 2,500 grams of ethanol per kilogram of catalyst per hour or from 600 to 2,000 grams of ethanol per kilogram of catalyst per hour.

Operating under the conditions of the present invention may result in ethanol production on the order of at least 0.1 tons of ethanol per hour, e.g., at least 1 ton of ethanol per hour, at least 5 tons of ethanol per hour, or at least 10 tons of ethanol per hour. Larger scale industrial production of ethanol, depending on the scale, generally should be at least 1 ton of ethanol per hour, e.g., at least 15 tons of ethanol per hour or at least 30 tons of ethanol per hour. In terms of ranges, for large scale industrial production of ethanol, the process of the present invention may produce from 0.1 to 160 tons of ethanol per hour, e.g., from 15 to 160 tons of ethanol per hour or from 30 to 80 tons of ethanol per hour. Ethanol production from fermentation, due the economies of scale, typically does not permit the single facility ethanol production that may be achievable by employing embodiments of the present invention.

In various embodiments of the present invention, the crude ethanol product produced by the hydrogenation process, before any subsequent processing, such as purification and separation, will typically comprise unreacted acetic acid, ethanol and water. As used herein, the term “crude ethanol product” refers to any composition comprising from 5 to 70 wt. % ethanol and from 5 to 40 wt. % water. Exemplary compositional ranges for the crude ethanol product are provided in Table 1. The “others” identified in Table 1 may include, for example, esters, ethers, aldehydes, ketones, alkanes, and carbon dioxide.

TABLE 1
CRUDE ETHANOL PRODUCT COMPOSITIONS

| | Conc. | Conc. | Conc. | Conc. |
|---|---|---|---|---|
| Component | (wt. %) | (wt. %) | (wt. %) | (wt. %) |
| Ethanol | 5 to 70 | 15 to 70 | 15 to 50 | 25 to 50 |
| Acetic Acid | 0.1 to 90 | 1 to 50 | 15 to 70 | 20 to 70 |
| Water | 5 to 40 | 5 to 30 | 10 to 30 | 10 to 26 |
| Ethyl Acetate | 0 to 30 | 0 to 20 | 1 to 12 | 3 to 10 |
| Acetaldehyde | 0 to 10 | 0 to 3 | 0.1 to 3 | 0.2 to 2 |
| Others | 0.1 to 10 | 0.1 to 6 | 0.1 to 4 | — |

In one embodiment, the crude ethanol product comprises acetic acid in an amount less than 20 wt. %, e.g., less than 15 wt. %, less than 10 wt. % or less than 5 wt. %. In embodiments having lower amounts of acetic acid, the conversion of acetic acid is preferably greater than 75%, e.g., greater than 85% or greater than 90%. In addition, the selectivity to ethanol may also be preferably high, and is preferably greater than 75%, e.g., greater than 85% or greater than 90%.

In one embodiment, the crude ethanol product may comprise acetic acid in an amount less than 20 wt. %, e.g., less than 15 wt. %, less than 10 wt. % or less than 5 wt. %. Of course, in order to recover acetic acid for recycle to the hydrogenation reactor according to embodiments of the present invention, some appreciable amount of acetic acid should be present in the crude ethanol product. In embodiments having lower amounts of acetic acid, the conversion of acetic acid is preferably greater than 50%, e.g., greater than 75% or greater than 90%. In addition, the selectivity to ethanol may also be preferably high, and is greater than 50%, e.g., greater than 75% or greater than 90%.

### Ethanol Recovery

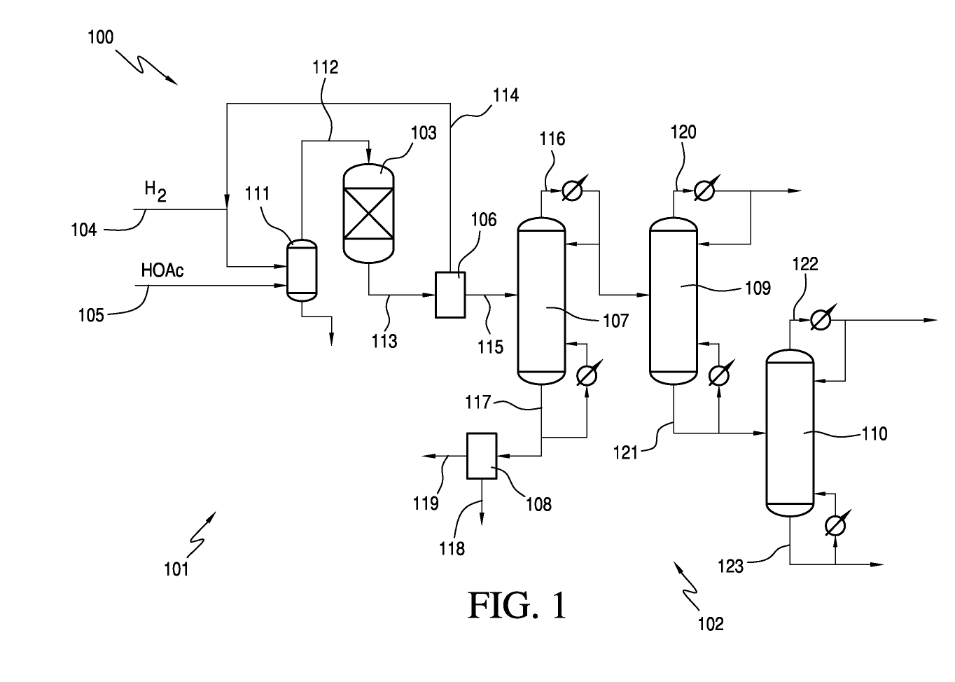

Exemplary ethanol recovery systems in accordance with embodiments of the present invention is shown in FIG. 1. System 100 comprises reaction zone 101 and separation zone 102. Reaction zone 101 comprises reactor 103, hydrogen feed line 104 and acetic acid feed line 105. Separation zone 102 comprises a separator 106, a first column 107, a water separator 108 and a second column 109.

Hydrogen and acetic acid are fed to a vaporizer 111 via lines 104 and 105, respectively, to create a vapor feed stream in line 112 that is directed to reactor 103. In one embodiment, lines 104 and 105 may be combined and jointly fed to the vaporizer 111, e.g., in one stream containing both hydrogen and acetic acid. The temperature of the vapor feed stream in line 112 is preferably from 100° C. to 350° C., e.g., from 120° C. to 310° C. or from 150° C. to 300° C. Any feed that is not vaporized is removed from vaporizer 111, as shown in FIG. 1, and may be recycled or discarded. In addition, although FIG. 1 shows line 112 being directed to the top of reactor 103, line 112 may be directed to the side, upper portion, or bottom of reactor 103. Further modifications and additional components to reaction zone 101 and separation zone 102 are described below.

Reactor 103 contains the catalyst that is used in the hydrogenation of the carboxylic acid, preferably acetic acid, to ethanol. In one embodiment, one or more guard beds (not shown) may be used upstream of the reactor, optionally upstream of vaporizer 111, to protect the catalyst from poisons or undesirable impurities contained in the feed or return/recycle streams. Such guard beds may be employed in the vapor or liquid streams. Suitable guard bed materials may include, for example, carbon, silica, alumina, ceramic, or resins. In one aspect, the guard bed media is functionalized, e.g., silver functionalized, to trap particular species such as sulfur or halogens. During the hydrogenation process, a crude ethanol product stream is withdrawn, preferably continuously, from reactor 103 via line 113.

The crude ethanol product stream in line 113 may be condensed and fed to a first separator 106, which, in turn, provides a vapor stream 114 and a liquid stream 115. Suitable separators 106 include a flasher or a knockout pot. The separator 106 may operate at a temperature of from 20° C. to 250° C., e.g., from 30° C. to 225° C. or from 60° C. to 200° C. The pressure of separator 106 may be from 50 kPa to 2000 kPa, e.g., from 75 kPa to 1500 kPa or from 100 kPa to 1000 kPa. In another embodiment, the temperature and/or pressure of the separator 106 may be similar to the temperature and/or pressure of the reactor 103. Optionally, the crude ethanol product in line 113 may pass through one or more membranes to separate hydrogen and/or other non-condensable gases.

The vapor stream 114 exiting separator 106 may comprise hydrogen and hydrocarbons, which may be purged and/or returned to reaction zone 101. As shown, vapor stream 114 is combined with the hydrogen feed 104 and co-fed to vaporizer 111. In some embodiments, the returned vapor stream 114 may be compressed before being combined with hydrogen feed 104.

The liquid stream 115 from separator 106 is withdrawn and pumped to the side of distillation column 107. In one embodiment, the contents of liquid stream 115 are substantially similar to the crude ethanol product obtained from the reactor in line 113, except that the composition has been depleted of hydrogen, carbon dioxide, methane and/or ethane, which are preferably removed by separator 106. Accordingly, liquid stream 115 may also be referred to as a crude ethanol product. Exemplary components of liquid stream 115 are provided in Table 2. It should be understood that liquid stream 115 may contain other components, not listed, such as components derived from the feed.

TABLE 2
COMPOSITION OF LIQUID STREAM 115

| | Conc. (wt. %) | Conc. (wt. %) | Conc. (wt. %) |
|---|---|---|---|
| Ethanol | 5 to 70 | 10 to 60 | 15 to 50 |
| Acetic Acid | 0.1 to 90 | 5 to 80 | 15 to 70 |
| Water | 5 to 40 | 5 to 30 | 10 to 30 |
| Ethyl Acetate | <30 | 0.001 to 20 | 1 to 12 |
| Acetaldehyde | <10 | 0.001 to 3 | 0.1 to 3 |
| Acetal | <5 | 0.001 to 2 | 0.005 to 1 |
| Acetone | <5 | 0.0005 to 0.05 | 0.001 to 0.03 |
| Other Esters | <5 | <0.005 | <0.001 |
| Other Ethers | <5 | <0.005 | <0.001 |
| Other Alcohols | <5 | <0.005 | <0.001 |

The amounts indicated as less than (<) in the tables throughout present application are preferably not present and if present may be present in trace amounts or in amounts greater than 0.0001 wt. %.

The “other esters” in Table 2 may include, but are not limited to, ethyl propionate, methyl acetate, isopropyl acetate, n-propyl acetate, n-butyl acetate or mixtures thereof. The “other ethers” in Table 2 may include, but are not limited to, diethyl ether, methyl ethyl ether, isobutyl ethyl ether or mixtures thereof. The “other alcohols” in Table 2 may include, but are not limited to, methanol, isopropanol, n-propanol, n-butanol or mixtures thereof. In one embodiment, the liquid stream 115, may comprise propanol, e.g., isopropanol and/or n-propanol, in an amount from 0.001 to 0.1 wt. %, from 0.001 to 0.05 wt. % or from 0.001 to 0.03 wt. %. It should be understood that these other components may be carried through in any of the distillate or residue streams described herein and will not be further described herein, unless indicated otherwise.

Optionally, crude ethanol product in line 113 or in liquid stream 115 may be further fed to an esterification reactor, a hydrogenolysis reactor, or combination thereof. An esterification reactor may be used to consume acetic acid present in the crude ethanol product to further reduce the amount of acetic acid to be removed. Hydrogenolysis may be used to convert ethyl acetate in the crude ethanol product to ethanol.

In the embodiment shown in FIG. 1, line 115 is introduced in the lower part of first column 107, e.g., lower half or lower third. Depending on the composition of the crude ethanol product in line 115 and the operating conditions of first column 107, the first column 107 separates the crude ethanol product in line 115, preferably continuously, into a first distillate in line 116 and a first residue in line 117. The first distillate in line 116 comprises ethanol, other organics and water. The first residue in line 117 comprises unreacted acetic acid, water, and other heavy components, if present. In some embodiments, especially with higher conversions of acetic acid of at least 80%, or at least 90%, it may be beneficial to remove a majority of the water, e.g., at least 50% of the water, from liquid stream 115, in the first residue along with substantially all of the acetic acid.

When column 107 is operated under about 170 kPa, the temperature of the residue exiting in line 117 preferably is from 90° C. to 130° C., e.g., from 95° C. to 120° C. or from 100° C. to 115° C. The temperature of the distillate exiting in line 116 preferably is from 60° C. to 90° C., e.g., from 65° C. to 85° C. or from 70° C. to 80° C. In some embodiments, the pressure of first column 107 may range from 0.1 kPa to 510 kPa, e.g., from 1 kPa to 475 kPa or from 1 kPa to 375 kPa. Exemplary components of the distillate and residue compositions for first column 107 are provided in Table 3 below. It should also be understood that the distillate and residue may also contain other components, not listed in Table 3. For convenience, the distillate and residue of the first column may also be referred to as the “first distillate” or “first residue.” The distillates or residues of the other columns may also be referred to with similar numeric modifiers (second, third, etc.) in order to distinguish them from one another, but such modifiers should not be construed as requiring any particular separation order.

TABLE 3
FIRST COLUMN 107

| | Conc. (wt. %) | Conc. (wt. %) | Conc. (wt. %) |
|---|---|---|---|
| First Distillate | | | |
| Ethanol | 20 to 90 | 30 to 85 | 50 to 85 |
| Water | 4 to 35 | 5 to 30 | 6 to 25 |
| Acetic Acid | <1 | 0.001 to 1 | 0.01 to 0.5 |
| Ethyl Acetate | <60 | 5 to 40 | 8 to 45 |
| Acetaldehyde | <10 | 0.001 to 5 | 0.01 to 4 |
| Acetal | <4.0 | <3.0 | <2.0 |
| Acetone | <0.05 | 0.001 to 0.03 | 0.01 to 0.025 |
| First Residue | | | |
| Acetic Acid | <98 | 40 to 95 | 50 to 90 |
| Water | <99.5 | 0.5 to 60 | 2 to 35 |
| Ethanol | <1 | <0.9 | <0.5 |

Some species, such as acetals, may decompose in column 107 such that very low amounts, or even no detectable amounts, of acetals remain in the distillate or residue. In addition, an equilibrium reaction between acetic acid and ethanol or between ethyl acetate and water may occur in the crude ethanol product after it exits reactor 103. Depending on the concentration of acetic acid in the crude ethanol product, this equilibrium may be driven toward formation of ethyl acetate. This equilibrium may be regulated using the residence time and/or temperature of crude ethanol product.

The first residue in line 117 comprises water, as discussed above, in addition to acetic acid and other heavy components. As shown in FIG. 1, at least a portion of the first residue in line 117 is fed to a water separator 108. Water separator 108 may comprise one or more adsorption units, membranes, molecular sieves, extractive column distillations, or a combination thereof. Adsorption units, such as pressure swing adsorption units or thermal swing adsorption units, are less preferred because of the acetic acid concentration in the first residue. A membrane is a preferred option for embodiments of the present invention. Water separator 108 may remove at least 50% of the water from the first residue in line 117, and more preferably from 90% to 99% of the water from the first residue, in a water stream 118. All or a portion of water stream 118 may be purged. The remaining portion of first residue 117 exits the water separator 108 as a return stream 119. Return stream 119 comprises acetic acid and preferably less than 15 wt. %, less than 10 wt. % water, or less than 5 wt. % water. In one embodiment, the concentrated acetic acid stream in return stream 119 may be returned to reactor 103. In other embodiments, depending on the composition, return stream 119 may be used in other production facilities for vinyl acetate, acetic anhydride, or ethyl acetate.

The first column 107 also forms an overhead distillate, which is withdrawn in line 116, and which may be condensed and refluxed, for example, at a ratio of from 10:1 to 1:10, e.g., from 3:1 to 1:3 or from 1:2 to 2:1. The final ethanol product may be derived from the distillate in line 116. In one embodiment, the weight ratio of water in the residue to the water in the distillate is greater than 1:1, e.g., greater than 2:1 or greater than 4:1. In addition, the weight ratio of acetic acid in the residue to acetic acid in the distillate is optionally greater than 10:1, e.g., greater than 15:1 or greater than 20:1. Preferably, the distillate in line 116 is substantially free of acetic acid and may contain, if any, only trace amounts of acetic acid.

Depending on the composition of the distillate in line 116, one or more further columns or separation units may be used to recover an ethanol product having a reduced water content from the distillate in line 116. The columns shown in FIG. 1 may comprise any distillation column capable of performing the desired separation and/or purification. Each column preferably comprises a tray column having from 1 to 150 trays, e.g., from 10 to 100 trays, from 20 to 95 trays or from 30 to 75 trays. The trays may be sieve trays, fixed valve trays, movable valve trays, or any other suitable design known in the art. In other embodiments, a packed column may be used. For packed columns, structured packing or random packing may be employed. The trays or packing may be arranged in one continuous column or they may be arranged in two or more columns such that the vapor from the first section enters the second section while the liquid from the second section enters the first section, etc.

The associated condensers and liquid separation vessels that may be employed with each of the distillation columns may be of any conventional design and are simplified in the figures. Heat may be supplied to the base of each column or to a circulating bottom stream through a heat exchanger or reboiler. Other types of reboilers, such as internal reboilers, may also be used. The heat that is provided to the reboilers may be derived from any heat generated during the process that is integrated with the reboilers or from an external source such as another heat generating chemical process or a boiler. Although one reactor and one flasher are shown in the figures, additional reactors, flashers, condensers, heating elements, and other components may be used in various embodiments of the present invention. As will be recognized by those skilled in the art, various condensers, pumps, compressors, reboilers, drums, valves, connectors, separation vessels, etc., normally employed in carrying out chemical processes may also be combined and employed in the processes of the present invention.

The temperatures and pressures employed in the columns may vary. As a practical matter, pressures from 10 kPa to 3000 kPa will generally be employed in these zones although in some embodiments subatmospheric pressures or superatmospheric pressures may be employed. Temperatures within the various zones will normally range between the boiling points of the composition removed as the distillate and the composition removed as the residue. As will be recognized by those skilled in the art, the temperature at a given location in an operating distillation column is dependent on the composition of the material at that location and the pressure of column In addition, feed rates may vary depending on the size of the production process and, if described, may be generically referred to in terms of feed weight ratios.

The first distillate in line 116 is introduced to a second column 109, also referred to as the “light ends column,” preferably in the top part of column 109, e.g., top half or top third. Second column 109 may be a tray column or packed column In one embodiment, second column 109 is a tray column having from 5 to 70 trays, e.g., from 15 to 50 trays or from 20 to 45 trays. As one example, when a 30 tray column is utilized in a column without water extraction, ethanol mixture stream 120 is introduced at tray 2.

Optionally, the light ends column may be an extractive distillation column Suitable extractive agents may include, for example, dimethylsulfoxide, glycerine, diethylene glycol, 1-naphthol, hydroquinone, N,N′-dimethylformamide, 1,4-butanediol; ethylene glycol-1,5-pentanediol; propylene glycol-tetraethylene glycol-polyethylene glycol; glycerine-propylene glycol-tetraethylene glycol-1,4-butanediol, ethyl ether, methyl formate, cyclohexane, N,N-dimethyl-1,3-propanediamine, N,N′-dimethylethylenediamine, diethylene triamine, hexamethylene diamine and 1,3-diaminopentane, an alkylated thiopene, dodecane, tridecane, tetradecane, chlorinated paraffins, or a combination thereof. In another aspect, the extractive agent may comprise water. If the extraction agent comprises water, the water may be obtained from an external source or from an internal return/recycle line from one or more of the other columns, such as from first residue 116 or a derivative stream thereof. Generally, the extractive agent is fed above the entry point of ethanol mixture stream 120, as shown by optional line 121. When extractive agents are used, a suitable recovery system, such as a further distillation column, may be used to remove the extractive agent and recycle the extractive agent if desired.

Second column 109 is operated to separate first distillate 116 or a portion thereof into a second distillate in line 120 and a second residue in line 121. Second distillate in line 121 may comprise, for example, ethyl acetate and acetaldehyde, while second residue comprises ethanol, which optionally is a salable finished ethanol product. Although the temperature and pressure of second column 109 may vary, when at atmospheric pressure the temperature of the second residue exiting in line 121 from second column 109 preferably is from 60° C. to 90° C., e.g., from 70° C. to 90° C. or from 80° C. to 90° C. The temperature of the second distillate exiting in line 120 from second column 109 preferably is from 50° C. to 80° C., e.g., from 60° C. to 80° C. or from 60° C. to 70° C. Column 109 may operate at a reduced pressure, near or at vacuum conditions, to further favor separation of ethyl acetate and ethanol. In some embodiments, for example, the pressure of second column 109 ranges from 0.1 kPa to 510 kPa, e.g., from 1 kPa to 475 kPa or from 1 kPa to 375 kPa.

Exemplary distillate and residue compositions for second column 109 are provided in Table 4, below. It should be understood that the second distillate and second residue may also contain other components, not listed in Table 4.

TABLE 4
SECOND COLUMN

| | Conc. (wt. %) | Conc. (wt. %) | Conc. (wt. %) |
|---|---|---|---|
| Second Distillate | | | |
| Ethanol | 5 to 90 | 0.001 to 15 | 0.01 to 5 |
| Ethyl Acetate | 1 to 90 | 2 to 90 | 3 to 80 |
| Acetaldehyde | 0.011 to 30 | 0.01 to 25 | 0.1 to 20 |
| Water | 0.1 to 20 | 0.5 to 15 | 1 to 10 |
| Acetal | <15 | 0.001 to 15 | 0.01 to 10 |
| Second Residue | | | |
| Water | 2 to 40 | 5 to 35 | 10 to 30 |
| Ethanol | 60 to 100 | 65 to 95 | 70 to 90 |
| Ethyl Acetate | <3 | 0.001 to 2 | 0.001 to 0.5 |
| Acetic Acid | <0.5 | 0.001 to 0.3 | 0.001 to 0.2 |

Any of the compounds that are carried through the distillation process from the feed or crude reaction product generally remain in the ethanol distillate in amounts of less 0.1 wt. %, based on the total weight of the ethanol distillate composition, e.g., less than 0.05 wt. % or less than 0.02 wt. %. In one embodiment, not shown, the ethanol product column may also form one or more side draw streams for removing impurities. The impurities may be purged and/or retained within the system 100.

The weight ratio of ethanol in the second residue to ethanol in the second distillate preferably is at least 2:1, e.g., at least 5:1, at least 8:1, at least 10:1 or at least 15:1. The weight ratio of ethyl acetate in the second residue to ethyl acetate in the second distillate preferably is less than 0.4:1, e.g., less than 0.2:1 or less than 0.1:1.

Second distillate in line 120, which comprises ethyl acetate and/or acetaldehyde, preferably is refluxed as shown in FIG. 1, for example, at a reflux ratio of from 1:30 to 30:1, e.g., from 1:5 to 5:1 or from 1:3 to 3:1. In some embodiments, the second distillate in line 120 or a portion thereof may be returned reactor 103. In some embodiments, it may be advantageous to return a portion of the second distillate to reactor 103. The ethyl acetate and/or acetaldehyde in the second distillate may be further reacted in hydrogenation reactor 103 or in a secondary reactor. The outflow from the secondary reactor may be fed to reactor 103 to produce additional ethanol or to a distillation column, such as columns, 107, 109, or 110, to recover additional ethanol.

Second residue in line 121 comprises ethanol. Depending on the intended ethanol application, it may be desirable to remove water, if any, from the second distillate. In some embodiments, removing substantially all of the water produces an anhydrous ethanol product suitable for fuel applications. Water may be removed from the distillate in line 121 using any of several different separation techniques. Particularly preferred techniques include the use of a distillation column, one or more membranes, one or more adsorption units or a combination thereof.

As shown, the second residue in line 121 is fed to a third column 110, also referred to as an ethanol product column, for separating the second residue into an ethanol distillate (third distillate) in line 122 and a water residue (third residue) in line 123. The second residue in line 121 may be introduced into the lower part of third column 110, e.g., lower half or lower third. The ethanol distillate 122 from the third column 110 preferably is refluxed, for example, at a reflux ratio of from 1:10 to 10:1, e.g., from 1:3 to 3:1 or from 1:2 to 2:1. Water residue in line 123 preferably is removed from the system.

Third column 110 is preferably a tray column as described above and preferably operates at atmospheric pressure or below. The temperature of the ethanol distillate exiting in line 122 preferably is from 60° C. to 110° C., e.g., from 70° C. to 100° C. or from 75° C. to 95° C. The temperature of the third residue 123 preferably is from 70° C. to 115° C., e.g., from 80° C. to 110° C. or from 85° C. to 105° C., when the column is operated at atmospheric pressure.

Exemplary compositions for the third distillate 122 and third residue 123 are provided below in Table 5. It should be understood that the third distillate and the third residue may also contain other components, not listed in Table 5.

TABLE 5
THIRD COLUMN 110

| | Conc. (wt. %) | Conc. (wt. %) | Conc. (wt. %) |
|---|---|---|---|
| Third Distillate | | | |
| Ethanol | 75 to 96 | 80 to 96 | 85 to 96 |
| Water | <12 | 1 to 9 | 3 to 8 |
| Acetic Acid | <1 | 0.001 to 0.1 | 0.005 to 0.01 |
| Ethyl Acetate | <5 | 0.001 to 4 | 0.01 to 3 |
| Third Residue | | | |
| Water | 75 to 100 | 80 to 100 | 90 to 100 |
| Ethanol | <0.8 | 0.001 to 0.5 | 0.005 to 0.05 |
| Ethyl Acetate | <1 | 0.001 to 0.5 | 0.005 to 0.2 |
| Acetic Acid | <2 | 0.001 to 0.5 | 0.005 to 0.2 |

In the embodiment shown in FIG. 1, the finished ethanol product produced by the process of the present invention may be taken from the either the second residue in line 121 and/or third distillate in line 122. Advantageously, the finished ethanol product may be recovered using three columns according to the present invention. The finished ethanol product may be an industrial grade ethanol comprising from 75 to 96 wt. % ethanol, e.g., from 80 to 96 wt. % or from 85 to 96 wt. % ethanol, based on the total weight of the ethanol product. Exemplary finished ethanol compositional ranges are provided below in Table 6.

TABLE 6
FINISHED ETHANOL COMPOSITIONS

| Component | Conc. (wt. %) | Conc. (wt. %) | Conc. (wt. %) |
|---|---|---|---|
| Ethanol | 75 to 96 | 80 to 96 | 85 to 96 |
| Water | <12 | 1 to 9 | 3 to 8 |
| Acetic Acid | <1 | <0.1 | <0.01 |
| Ethyl Acetate | <2 | <0.5 | <0.05 |
| Acetal | <0.05 | <0.01 | <0.005 |
| Acetone | <0.05 | <0.01 | <0.005 |
| Isopropanol | <0.5 | <0.1 | <0.05 |
| n-propanol | <0.5 | <0.1 | <0.05 |

The finished ethanol composition of the present invention preferably contains very low amounts, e.g., less than 0.5 wt. %, of other alcohols, such as methanol, butanol, isobutanol, isoamyl alcohol and other C₄-C₂₀ alcohols. In one embodiment, the amount of isopropanol in the finished ethanol composition is from 80 to 1,000 wppm, e.g., from 95 to 1,000 wppm, from 100 to 700 wppm, or from 150 to 500 wppm. In one embodiment, the finished ethanol composition is substantially free of acetaldehyde, optionally comprising less than 8 wppm acetaldehyde, e.g., less than 5 wppm or less than 1 wppm.

In some embodiments, second residue in line 121 and/or third distillate in line 122 may be further treated to remove additional water in a secondary water separation unit. Residual water removal may be accomplished, for example, using an adsorption unit, membrane, molecular sieves, extractive column distillation, or a combination thereof. In such embodiments, the ethanol concentration of the finished ethanol product after the secondary water removal step may be greater than the values indicated in Table 6. In this embodiment, the ethanol concentration of the ethanol product may be greater than 97 wt. %, e.g., greater than 98 wt. % or greater than 99.5 wt. %. The ethanol product in this aspect preferably comprises less than 3 wt. % water, e.g., less than 2 wt. % or less than 0.5 wt. %.

The finished ethanol composition produced by the embodiments of the present invention may be used in a variety of applications including applications as fuels, solvents, chemical feedstocks, pharmaceutical products, cleansers, sanitizers, hydrogenation transport or consumption. In fuel applications, the finished ethanol composition may be blended with gasoline for motor vehicles such as automobiles, boats and small piston engine aircraft. In non-fuel applications, the finished ethanol composition may be used as a solvent for toiletry and cosmetic preparations, detergents, disinfectants, coatings, inks, and pharmaceuticals. The finished ethanol composition may also be used as a processing solvent in manufacturing processes for medicinal products, food preparations, dyes, photochemicals and latex processing.

The finished ethanol composition may also be used as a chemical feedstock to make other chemicals such as vinegar, ethyl acrylate, ethyl acetate, ethylene, glycol ethers, ethylamines, aldehydes, and higher alcohols, especially butanol. In the production of ethyl acetate, the finished ethanol composition may be esterified with acetic acid. In another application, the finished ethanol composition may be dehydrated to produce ethylene. Any known dehydration catalyst can be employed to dehydrate ethanol, such as those described in copending U.S. Pub. Nos. 2010/0030002 and 2010/0030001, the entire contents and disclosures of which are hereby incorporated by reference. A zeolite catalyst, for example, may be employed as the dehydration catalyst. Preferably, the zeolite has a pore diameter of at least about 0.6 nm, and preferred zeolites include dehydration catalysts selected from the group consisting of mordenites, ZSM-5, a zeolite X and a zeolite Y. Zeolite X is described, for example, in U.S. Pat. No. 2,882,244 and zeolite Y in U.S. Pat. No. 3,130,007, the entireties of which are hereby incorporated herein by reference.

In order that the invention disclosed herein may be more efficiently understood, an example is provided below. It should be understood that this example is for illustrative purposes only and is not to be construed as limiting the invention in any manner.

### Example

The following examples were prepared with ASPEN Plus 7.1 simulation software to test various feed composition and separation systems.

Reactor product comprising 32.5 wt. % ethanol, 31 wt. % water, 26 wt. % ethyl acetate, 0.3 wt. % acetaldehyde, 10 wt. % acetic acid, and 0.2 wt. % other organics were into a distillation columns The distillation column operated with 40 trays at 1 atmospheric pressure. The feed tray was located at 15^{th} tray from the top. Run A was operated with a higher energy than Run B and the distillate and residue compositions are shown in Table 7 as observed using ASPEN simulation.

TABLE 7

| | Run A | Run B |
|---|---|---|
| Distillate - wt. % | | |
| Ethanol | 36.1 | 49.2 |
| Water | 33.4 | 10.6 |
| Ethyl Acetate | 28.9 | 39.4 |
| Acetaldehyde | 0.3 | <0.01 |
| Other organics | 0.3 | 0.4 |
| Residue - wt. % | | |
| Ethanol | <0.01 | <0.01 |
| Water | 9.2 | 70.6 |
| Acetic Acid | 90.8 | 29.4 |
| Energy (MMBtu/ton ETOH) | 8.2 | 2.3 |

The water removal from residue stream from Run B using water permeable membrane was calculated. The water permeate comprised 98 wt. % water, and 2 wt. % acetic acid. The water permeate also comprised less than 0.01 wt. % of ethanol, ethyl acetate, and/or acetaldehyde. The water removal from the residue stream was greater than 99%.

While the invention has been described in detail, modifications within the spirit and scope of the invention will be readily apparent to those of skill in the art. In addition, it should be understood that aspects of the invention and portions of various embodiments and various features recited herein and/or in the appended claims may be combined or interchanged either in whole or in part. In the foregoing descriptions of the various embodiments, those embodiments which refer to another embodiment may be appropriately combined with one or more other embodiments, as will be appreciated by one of skill in the art. Furthermore, those of ordinary skill in the art will appreciate that the foregoing description is by way of example only, and is not intended to limit the invention.

We claim:

1. A process for producing ethanol, comprising:
hydrogenating acetic acid in a reactor to form a crude ethanol product comprising ethanol, acetic acid, water and ethyl acetate;
separating a portion of the crude ethanol product to yield a first stream comprising ethanol, ethyl acetate, and water, and a second stream comprising acetic acid and water;
removing water from the second stream to yield a return stream comprising acetic acid and less than 15 wt. % water; and
recovering ethanol from the first stream.

2. The process of claim 1, wherein the return stream comprises less than 5 wt. % water.

3. The process of claim 1, wherein at least 50% of the water in the second stream is removed as a water stream.

4. The process of claim 1, wherein from 90 to 99% of the water in the second stream is removed in the removing step.

5. The process of claim 1, wherein the second stream comprises from 40 wt. % to 95 wt. % acetic acid and from 0.5 wt. % to 60 wt. % water.

6. The process of claim 1, wherein at least a portion of the return stream is fed to the reactor.

7. The process of claim 1, wherein at least a portion of the return stream is used in the production of vinyl acetate, acetic anhydride, or ethyl acetate.

8. The process of claim 1, wherein water is removed from the second stream using a membrane.

9. The process of claim 1, wherein the first stream comprises ethanol in an amount from 50 to 85 wt. %, water in an amount from 6 to 25 wt. %, ethyl acetate in an amount from 8 to 45 wt. %, and acetaldehyde in an amount from 0.01 to 4 wt. %.

10. The process of claim 1, wherein the acetic acid is formed from methanol and carbon monoxide, wherein each of the methanol, the carbon monoxide, and hydrogen for the hydrogenating step is derived from syngas, and wherein the syngas is derived from a carbon source selected from the group consisting of natural gas, oil, petroleum, coal, biomass, and combinations thereof.

11. A process for producing ethanol, comprising:
hydrogenating acetic acid to form a crude ethanol product;
separating a portion of the crude ethanol product in a first distillation column to yield a first distillate comprising ethanol, ethyl acetate, and water; and a first residue comprising acetic acid;
removing water from the first residue to form a return stream comprising acetic acid; and
separating a portion of the first distillate in a second distillation column to yield a second residue comprising ethanol and a second distillate comprising ethyl acetate.

12. The process of claim 11, wherein at least 50% of the water in the first residue is removed in the removing step.

13. The process of claim 11, wherein the first residue comprises from 40 wt. % to 95 wt. % acetic acid and from 0.5 wt. % to 60 wt. % water.

14. The process of claim 11, wherein the return stream is fed to the reactor.

15. The process of claim 11, wherein at least a portion of the return stream is used in the production of vinyl acetate, acetic anhydride, or ethyl acetate.

16. The process of claim 11, wherein water is removed from the first residue using a membrane.

17. The process of claim 11, further comprising reducing the water content of the second residue to yield an ethanol product stream with reduced water content.

18. A process for recovering ethanol, comprising:
providing a crude ethanol product comprising ethanol, acetic acid, ethyl acetate, and water;
separating a portion of the crude ethanol product to yield a first stream comprising ethanol, ethyl acetate, and water, and a second stream comprising acetic acid and water;
removing water from the second stream to yield a return stream comprising acetic acid and less than 15 wt. % water; and
recovering ethanol from the first stream.

19. The process of claim 18, wherein the return stream comprises less than 15 wt. % water.

20. The process of claim 18, wherein water is removed from the second stream using a membrane.
